(19) United States
(12) Patent Application Publication
(10) Pub. No.: US 2026/0151462 A1
(43) Pub. Date: Jun. 4, 2026

(54) RECOMBINANT APYRASE PROTEIN FOR USE IN THE TREATMENT OF AN ISCHEMIC EVENT AT A DOSE OF 40-240 MG

(71) Applicant: ASTRAZENECA AB, Södertälje (SE)

(72) Inventors: Ann-Charlotte Eva EGNELL, Gothenberg (SE); Anna Marie ELEBRING, Gothenberg (SE); Björn Magnus ÅSTRAND, Gothenberg (SE); Peter Daniel HOVDAL, Gothenberg (SE)

(21) Appl. No.: 19/123,818

(22) PCT Filed: Oct. 26, 2023
(86) PCT No.: PCT/EP2023/079971

§ 371 Date: Apr. 24, 2025

Related U.S. Application Data
(60) Provisional application No. 63/381,363, filed on Oct. 28, 2022.

(51) Int. Cl. A61K 38/46; A61K 9/00; A61K 31/616; A61K 31/7064; A61P 9/10

(52) U.S. Cl. CPC A61K 38/46; A61K 9/0019; A61K 31/616; A61K 31/7064; A61P 9/10; C12Y 306/01005

(57) ABSTRACT

The present disclosure relates to dosage regimens for the administration of a recombinant apyrase protein and their medical use in the treatment of ischemic events in a patient, such as ST-segment elevation myocardial infarction and acute ischemic stroke.

### FIELD

The present disclosure relates to dosage regimens for the administration of a recombinant apyrase protein and their medical use in the treatment of ischemic events in a patient, such as ST-segment elevation myocardial infarction and acute ischemic stroke. The recombinant apyrase protein may be administered in conjunction with a dual antiplatelet therapy comprising a P2Y₁₂ inhibitor and aspirin.

### BACKGROUND

Myocardial infarction (MI) is a leading cause of hospital admissions and mortality around the world (Asaria et al. 2017). If left untreated, MI results in irreversible damage to the heart muscle due to a lack of blood flow (ischaemia) and thus oxygen. A primary goal of therapy with MI is therefore to expedite restoration of normal coronary blood flow with the aim of decreasing heart muscle damage through reperfusion therapy. Reperfusion therapy typically involves the use of therapeutics to increase blood flow and reduce thrombosis combined with surgical techniques such as percutaneous coronary intervention (PCI). Early reperfusion and PCI is preferable and associated with improved outcomes, with Guidelines suggesting PCI should be performed within 12 hours of MI symptom onset (Ibanez et al. 2018).

One commonly used small molecule used for reducing thrombosis is aspirin. Another type of small molecule therapeutics is the P2Y₁₂ receptor inhibitors such as clopidogrel, ticagrelor, prasugrel and cangrelor, which are known for their ability to inhibit platelets and prevent blood clots. The combined administration of aspirin and P2Y₁₂ receptor inhibitors is referred to as dual antiplatelet therapy (DAPT), which has been shown to be clinically effective in the prevention of thrombotic events. For example, the National Institute for Health and Care Excellence (NICE) recommends ticagrelor in combination with low-dose aspirin for up to 12 months as a therapy for adults with acute coronary syndromes (ACS). These P2Y₁₂ receptor inhibitors do however come with an increased bleeding risk and therefore care does need to be taken to ensure that patients receive this treatment in accordance with the prescribing information and current guidelines.

Despite optimal anti-platelet and anti-thrombotic treatment involving P2Y₁₂ receptor inhibitors such as ticagrelor, there is still around a 10% yearly risk of a new myocardial infarction event and increasing the intensity of these treatments increases bleeding risk without improving efficacy (Wallentin et al., 2009; Jernberg et al., 2015). Accordingly, there remains a need for effective and safe therapeutic methods that can be used in the treatment of an ischemic event such as a myocardial infarction.

Recent studies have investigated the use of recombinant apyrases as a protein-based therapeutic. Apyrases (ecto-ATP diphosphohydrolases) constitute a group of enzymes catalysing metabolism of ATP to ADP and ADP to AMP. In the body, the AMP produced by apyrase-induced hydrolysis of ATP and ADP is converted into adenosine by the ubiquitously expressed extracellular CD73/ecto-5′-nucleotidase. The first known human apyrase, CD39, was originally identified as a cell-surface protein on activated lymphocytes and endothelial cells and various in vitro and in vivo studies have shown apyrase is able to maintain vascular integrity and physiologically inhibit inflammation and thrombosis (Robson et al. 2005).

Moeckel et al. (2014) reports on the design and production of a recombinant optimised form of soluble CD39L3, a member of the human CD39 family. The resulting recombinant protein is termed ‘APT102’ or ‘AZD3366’. The authors report that this recombinant protein exhibited four times higher adenosine diphosphatase activity and a 50 times longer plasma half-life than native apyrase and that treatment with APT102 in animal models decreased infarction size without an increase in bleeding time. WO 2022/038191 describes methods for treating ischemic events in a patient, such as ST-segment elevation myocardial infarction and acute ischemic stroke, by administering AZD3366 in conjunction with a P2Y₁₂ inhibitor.

Data on the behaviour, safety and efficacy of recombinant apyrases, including AZD3366, in human patients including dosages for administration, have not been available to date.

### SUMMARY

AZD3366 is a recombinant human apyrase engineered to enhance hydrolysis of extracellular adenosine tri-/diphosphate (ATP/ADP) to adenosine monophosphate and subsequently adenosine. In pre-clinical studies performed in dog, rodent and pig models (Moeckel et al. 2014 and unpublished results), AZD3366 has exhibited anti-thrombotic, anti-inflammatory and tissue protective properties. The present inventors sought to determine, for the first time, how AZD3366 behaves when administered to humans at various doses.

The inventors established through a human clinical trial that AZD3366 used alone or in combination with aspirin and ticagrelor was generally safe and well tolerated when administered to humans. Furthermore, the in vivo pharmacokinetic and safety data obtained from the in-human trial was combined with pre-clinical efficacy data obtained from animal models to predict dosages of AZD3366 that are expected to achieve an efficacious cardioprotective effect in humans without compromising patient safety. It was additionally established these doses achieved complete platelet inhibition without a significant increase in bleeding-elated adverse events when used in humans, demonstrating a further clinical benefit attributed to the administration of AZD3366 in treating ischemic events.

Hence, administering AZD3366 at the doses described herein is expected to achieve clinical benefit when administered to humans for the treatment of ischemic events, including the treatment of acute coronary syndromes such as ST-segment elevation myocardial infarction, and the treatment of acute ischemic stroke.

Accordingly, one aspect of the present disclosure provides a method of treating an ischemic event in a patient, the method comprising administering to the patient a therapeutically effective amount of a recombinant apyrase protein, wherein the method comprises administering the recombinant apyrase protein to the patient at a dose of 40 mg to 240 mg.

In another aspect the present disclosure provides a recombinant apyrase protein for use in a method of treating an ischemic event in a patient, wherein the method comprising administering the recombinant apyrase protein to the patient at a dose of 40 mg to 240 mg.

In another aspect, the present disclosure provides the use of a recombinant apyrase protein in the manufacture of a medicament for the treatment of an ischemic event in a patient, the treatment comprising administering the recombinant apyrase protein to the patient at a dose of 40 mg to 240 mg.

The recombinant apyrase protein may comprise the amino acid sequence set forth as SEQ ID NO: 2 (AZD3366).

The recombinant apyrase protein (e.g. AZD3366) may be administered to the patient at a dose of 40 mg to 170 mg, 40 mg to 150 mg, 40 mg to 140 mg or 40 to 100 mg.

The recombinant apyrase protein (e.g. AZD3366) may be administered to the patient at a dose of 100 mg to 240 mg, 100 mg to 220 mg, or 100 mg to 200 mg.

The recombinant apyrase protein (e.g. AZD3366) may be administered to the patient at a dose of 100 mg to 140 mg, such as at a dose of 100 mg, 105 mg, 110 mg, 115 mg, 120 mg, 125 mg, 130 mg, 135 mg, or 140 mg. In one embodiment, the recombinant apyrase protein (e.g. AZD3366) is administered to the patient at a dose of 115 mg.

The recombinant apyrase protein may be administered to the patient by intravenous injection.

In some embodiments, the ischemic event being treated is an acute coronary syndrome. Acute coronary syndromes include ST segment elevation myocardial infarction (STEMI), non-ST segment elevation myocardial infarction (NSTEMI) and unstable angina. In some embodiments, the acute coronary syndrome being treated is a ST segment elevation myocardial infarction (STEMI) in a patient. In other embodiments, the ischemic event being treated is acute ischemic stroke.

As demonstrated herein, AZD3366 can be safely administered to humans when used alone, or in combination with aspirin and ticagrelor. Hence, in some embodiments the recombinant apyrase protein is administered in conjunction with a P2Y₁₂ inhibitor and/or with aspirin. In some embodiments, the recombinant apyrase protein is administered in conjunction with a dual anti-platelet therapy (DAPT), where the DAPT comprises administration with both a P2Y₁₂ inhibitor and aspirin. A skilled physician or other skilled medical personnel can determine the most suitable manner of administering each therapeutic agent to the patient, with exemplary dosages and treatment regiments further described herein.

The P2Y₁₂ inhibitor may be selected from the group consisting of ticagrelor, clopidogrel, ticlopidine, prasugrel, and cangrelor. In some embodiments, P2Y₁₂ inhibitor is selected from the group consisting of ticagrelor, clopidogrel and prasugrel. In some embodiments, the P2Y₁₂ inhibitor is ticagrelor or clopidogrel. In some embodiments, the P2Y₁₂ inhibitor is ticagrelor.

Where the recombinant apyrase protein is administered in combination with a P2Y₁₂ inhibitor and/or aspirin, the different agents are typically administered as separate formulations that are administered sequentially. Where sequential administration is used, the recombinant apyrase protein may be administered within 12 hours, 6 hours or 2 hours of loading doses of the P2Y₁₂ inhibitor and/or aspirin being administered.

Following administration of the recombinant apyrase protein and loading doses of P2Y₁₂ inhibitor and/or aspirin being administered, further maintenance doses of the P2Y₁₂ inhibitor and aspirin (e.g. for at least 2 days, at least a week, at least 6 weeks, or at least 6 months). As demonstrated herein, while there was an increase in capillary bleeding time (CBT) observed in humans upon administration of a dose of AZD3366 with loading doses of ticagrelor and aspirin, that increase levelled off and became insignificant as maintenance doses of ticagrelor and aspirin continued.

The disclosure includes the combination of the aspects and preferred features described except where such a combination is clearly impermissible or expressly avoided.

### SUMMARY OF THE FIGURES

Embodiments and experiments illustrating the principles of the disclosure will now be discussed with reference to the accompanying figures in which:

FIG. 1 illustrates the percent platelet aggregation inhibition from baseline over time from humans administered with different doses (10 mg, 30 mg, 90 mg, 180 mg, 360 mg and 640 mg) of AZD3366 (mean with 1 SEM error bars). Platelet aggregation was quantified in Platelet Rich Plasma (PRP) using an Light Transmission Aggregometry (LTA) assay further described herein. Complete platelet inhibition at 10 mg dose and above with a dose-dependent duration was observed.

FIG. 2 illustrates the capillary bleeding time (CBT) data obtained from humans administered with different doses (2 mg, 10 mg, 30 mg, 90 mg, 180 mg, 360 mg and 640 mg) of AZD3366. No significant increase in CBT up to the 90 mg dose level was observed.

FIG. 3 illustrates the capillary bleeding time (CBT) data obtained from humans administered with AZD3366 in combination with aspirin (ASA) and ticagrelor (tica). Loading and maintenance doses of aspirin and ticagrelor were administered according to normal clinical practice, as further described in the examples. Baseline, A, B and C represent timepoints for CBT measurements. The baseline panel provides CBT measurements before administration of any of aspirin, AZD3366 or ticagrelor (0002:30 PRE, or 2 hours 30 minutes before AZD3366 administration). The A panel provides CBT measurements taken after administration of AZD3366 or placebo, and a loading dose of 324 mg aspirin (0000:10 POST, meaning CBT measurement taken 10 minutes after AZD3366 or placebo administration). The B panel provides CBT measurements after administration of AZD3366 or placebo, and loading doses of 324 mg aspirin and 180 mg ticagrelor (0003:40 POST, meaning CBT measurement taken 3 hours 40 minutes after AZD3366 or placebo administration). The C panel provides CBT measurements after administration of AZD3366 or placebo, loading doses of 324 mg aspirin and 180 mg ticagrelor and maintenance doses of 81 mg aspirin and 90 mg ticagrelor (0051:40 POST, meaning CBT measurement taken 51 hours 40 minutes after AZD3366 administration). There was a significant increase in CBT in the AZD3366 group compared to placebo when loading doses of ASA and ticagrelor were given but that increase levelled off when healthy volunteers continued with maintenance doses of DAPT

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

### Recombinant Apyrase Proteins

An apyrase (EC 3.6.1.5) catalyses the hydrolysis of phosphoanhydride bonds of adenosine triphosphate (ATP) to adenosine monophosphate (AMP) and adenosine diphosphate (ADP) to AMP. CD39 family members (also termed ecto-nucleoside triphosphate diphosphohydrolase (E-NTPDase) family members) represent some of the best characterised apyrases. Human CD39 family members include the native proteins set out in the following table:

| Name of native | | NCBI accession |
|---|---|---|
| protein | Additional names | number |
| CD39 | ATPDase, ecto-apyrase, | U87967.1 |
| | NTPDase1 | |
| CD39L1 | NTPDase2, ecto-ATPase | AF144748.1 |
| CD39L2 | NTPDase6 | AY327581.1 |
| CD39L3 | NTPDase3, CD39L3, HB6 | AF034840.2 |
| CD39L4 | NTPDase5, ER-UDPase, | AF039918.1 |
| | PCPH | |
| LALP70 | UDPase, NTPDase4 | AF016032.1 |
| LALP1 | NTPDase7 | AF269255.1 |
| liver canalicular | NTPDase8, hATPDase | AY430414.1 |
| ecto-ATPase | | |

Human CD39L3 is a 529 amino acid residue protein shown in SEQ ID NO: 1. A particular exemplary soluble CD39L3 apyrase protein has the amino acid sequence set forth as position 49-485 of SEQ ID NO: 1.

The design, production and use of soluble recombinant apyrase proteins including engineered versions of CD39L3 (e.g. enhanced apyrases) are described in U.S. Pat. No. 7,247,300B1, EP2133430B1 and EP2523971B1, all of which are incorporated herein by reference in their entireties. The recombinant apyrase protein described herein may be any of the apyrases described in those publications.

EP2133430B1 describes ADPase enhanced apyrases. These ADPase enhanced apryases comprise modified forms of reference apyrases, wherein the modification results in increased ADPase activity compared with the reference apyrase or the same ADPase activity as the reference apyrase combined with decreased ATPase activity as compared with the reference apyrase. Exemplary ADPase enhanced apyrases include those comprising substitutions at positions 67 and 69 of CD39L3, wherein the position numbering is in accordance with SEQ ID NO: 1. Specifically, ADPase enhanced apyrases include protein 8742, comprising R67G and T69R substitutions; and protein 8906, comprising R67A and T69R substitutions.

Exemplary ATPase and ADPase assays used to determine this activity are disclosed in EP2133430B1. For example, ATPase and ADPase enzyme activities of purified soluble ADPase enhanced apyrases can be determined at 37° C. in a 1 ml solution containing 8 mM CaCl2, 200 μM substrate (ATP for ATPase or ADP for ADPase), 50 mM imidazole, and 50 mM Tris, pH 7.5 (Picher, et al., Biochem. Pharmacol. (1938) 51: 1453). The reaction can be stopped and inorganic phosphate released can be measured by addition of 0.25 ml of malachite green reagent (Baykov, et al., Anal. Biochem. (1988) 171: 266). Based on the spectrophotometric analysis at 630 nm, one unit of ATPase (or ADPase) corresponds to release of 1 μmole of inorganic phosphate/min at 37° C. Key kinetic constants for the enzyme such as Km and kcat may be obtained by fitting data into, for example, a Michaelis-Menten equation. Other assays useful for monitoring biochemical function include, but are not limited to, a radiometric assay, a HPLC assay both described by Gayle III, et al. (J. Clin Invest. (1998) 101: 1851-1859) or a radio-TLC assay described by Marcus, A. J., et al. (J. Clin Invest. (1991) 88:1690-1696).

Thus, the recombinant apyrase protein described herein may comprises one or more modifications (e.g. amino acid substitutions) compared with a reference apyrase having the amino acid sequence set forth as positions 49-485 of SEQ ID NO: 1 (i.e. a soluble version of CD39L3). These modification(s) may result in increased ADPase activity compared with the reference apyrase or the same ADPase activity as the reference apyrase combined with decreased ATPase activity as compared with the reference apyrase. In some embodiments, the one or more modifications may comprise or consist of substitutions at positions 67 and 69, wherein the positions are numbered according to SEQ ID NO: 1. The substitution at position 67 may be to a glycine and the substitution at position 69 may be to an arginine, or the substitution at position 67 may be to an alanine and the substitution at position 69 may be to an arginine. In some embodiments, the substitution at position 67 is to a glycine and the substitution at position 69 is to an arginine.

EP2523971B1 describes apyrases and methods of producing apyrases that comprise a homogeneous N-terminus, e.g. such that more than 80% of the apyrase molecules have the same N-terminus which comprises EVLP. These proteins with a homogenous N-terminus are described as having an average isoelectric point in the range of 3.0 to 4.5 and/or having enhanced half lives in rabbits and pigs.

Thus, the recombinant apyrase protein described herein may comprise a homogenous N-terminus such that more than 80% of the apyrase molecules have the same N-terminus, which N-terminus is EVLP, as described in EP2523971B1.

In certain embodiments, the recombinant apyrase protein may further comprise one or more functionally conservative substitutions (e.g. in addition to the substitutions in the ADPase enhanced apyrases described above. Functionally conservative substitutions are substitutions that do not affect (or do not substantially affect) one or more functional properties (e.g. enzymatic activity) as compared to the equivalent unsubstituted protein. In some embodiments the recombinant apyrase protein comprises 1, 2, 3, 4, 5, 6, 7, 8, 9, 10, 11, 12, 13, 14, 15, 16, 17, 18, 19 or 20 functionally conservative substitutions.

In some embodiments, the recombinant apyrase protein comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 2, optionally wherein the amino acid residue at position 67 is a glycine and the amino acid residue at position 69 is an arginine, wherein the positions are numbered according to SEQ ID NO: 1. For example, the recombinant apyrase protein may comprise the amino acid sequence of SEQ ID NO: 2 with 1 or more (e.g. 1, 2, 3, 4, 5, 6, 7, 8, 9, 10, 11, 12, 13, 14, 15, 16, 17, 18, 19 or 20) functionally conservative substitutions, optionally wherein the amino acid residue at position 67 is a glycine and the amino acid residue at position 69 is an arginine, wherein the positions are numbered according to SEQ ID NO: 1.

In exemplary embodiments, the recombinant apyrase protein comprises the amino acid sequence of SEQ ID NO: 2 (AZD3366).

### Treatment of Ischemic Event

The methods and products for use described herein are for treating an ischemic event in a patient (e.g. a human patient), which includes diseases and disorders where the blood supply is restricted to a particular part of the patient's body, e.g. the patient's heart or brain, where the restriction may be caused by a blood clot (thrombus). Ischemic events in the heart include an acute coronary syndrome. Ischemic events in the brain include acute ischemic stroke (AIS). Acute coronary syndromes include myocardial infarctions classified as ST segment elevation myocardial infarction (STEMI) or non-ST segment elevation myocardial infarction (NSTEMI), and unstable angina. In some embodiments, the treatment is a treatment of a ST segment elevation myocardial infarction (STEMI) in a patient.

Myocardial infarctions are generally clinically classified into STEMI and NSTEMI. These are based on changes to an electrocardiogram (ECG) and can be diagnosed by a physician or other skilled medical personnel. The type of myocardial infarction may be as defined in accordance with or derived from the universal definition of myocardial infarction set out in Thygesen et al. 2018.

In some embodiments, the recombinant apyrase protein is administered to the patient 18 hours or less, or 12 hours or less, or 6 hours or less, or 4 hours or less, or 2 hours or less, or 1 hour or less, or even 30 minutes or less after onset of the ischemic event (e.g. acute coronary syndrome such as STEMI). Onset of the ischemic event as referred to herein may be at the onset of one or more symptoms of the ischemic event (e.g. chest pain in the case of STEMI) or at the time of diagnosis, (e.g. via electrocardiogram in the case of acute coronary syndrome), which may be carried out prior to or shortly after the patient arrives at a hospital (or equivalent thereof) for treatment.

In some embodiments, the patient is administered with the recombinant apyrase protein prior to surgical reperfusion therapy (e.g. percutaneous coronary intervention (PCI)) being performed on the patient suffering from the acute coronary syndrome.

PCI may comprise, without limitation, balloon angioplasty, stent implantation, rotational or laser atherectomy, and/or brachytherapy. In instances in which a stent is implanted, the stent may be, without limitation, a bare-metal stent, a drug-eluting stent, an absorbable stent, etc., as known in the art.

The cardioprotective effect provided by the P2Y₁₂ inhibitor may be useful to prevent and/or alleviate any injury to cardiac tissue or function attributed to the restoration of circulation following reperfusion therapy. Thus, in some embodiments the method further comprises carrying out surgical reperfusion therapy (e.g. PCI) on the patient less than 48 hours, less than 24 hours, less than 12 hours, less than 6 hours following administering the recombinant apyrase protein.

Treatment of the acute coronary syndrome may be revealed by a decrease in infarct size in the patients and/or by restoration of blood flow to the affected area. Additionally, treatment by the recombinant apyrase protein may result in inhibition of platelet aggregation in the patients. As demonstrated herein, complete platelet inhibition was achieved by AZD3366 at all the doses tested, with a dose dependent duration effect reported. Methods of monitoring the success of acute coronary syndrome treatment will be understood by the skilled practitioner, for example from their knowledge of treatment of acute coronary syndrome using known P2Y₁₂ inhibitors.

In other embodiments, the ischemic event being treated is acute ischemic stroke. Every year, 100 new patients have an ischaemic stroke in a population of 70 000. Without treatment, 55 patients die or become dependent within a year. Most of the 100 patients have mild or transient stroke and receive only antiplatelet drugs to reduce recurrence. About 25-35 patients receive reperfusion therapy, which saves 5-6 patients from death or dependency and increases numbers with no disability. Therefore, it is an unmet clinical need to reduce morbidity and mortality associated with acute ischaemic stroke (AIS).

Preclinical and clinical trials have evaluated the use of P2Y₁₂ inhibitors and aspirin to treat and/or prevent stroke after an ischemic stroke event. Furthermore, preclinical data has demonstrated that the recombinant apyrase protein AZD3366 can be used to enhance reperfusion, reduce re-occlusion and reduce intracerebral bleeding in animal models of ischemic stroke (Sun et al., 2011; Tan et al., 2014). It was therefore realised that the beneficial results in the preclinical myocardial infarction animal model described herein associated with administration of AZD3366 (optionally in combination with a P2Y₁₂ inhibitor and/or aspirin) could also be beneficial in the treatment of ischaemic stroke. For example, the treatment may reduce infarct size and/or reduce brain damage in patients suffering from ischaemic stroke and achieve this effect without a significantly increased risk of bleeding compared to P2Y₁₂ inhibitors alone.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

### Dosages and Treatment Regimens

Administration of the recombinant apyrase protein described herein may be given by, for example, bolus injection, intravenously, intramuscularly, subcutaneously, inhalation, continuous infusion, sustained release, or other pharmaceutically acceptable techniques. The recombinant apyrase protein described herein may be administered to the patient by intravenous injection, as was done in the AZD3366 Phase I study reported in the examples.

The recombinant apyrase protein may be administered to the patient at a dose of 20 mg to 640 mg. As described herein, doses of AZD3366 within this range were safe and well-tolerated when administered to humans.

In some cases, the recombinant apyrase protein may be administered to the patient at a dose of 40 mg to 240 mg, 50 mg to 240 mg, 60 mg to 240 mg, 70 mg to 240 mg, 80 mg to 240 mg, 90 mg to 240 mg, 100 mg to 240 mg, 110 mg to 240 mg, 120 mg to 240 mg, 130 mg to 240 mg, 140 mg to 240 mg, 150 mg to 240 mg, 160 mg to 240 mg, 170 mg to 240 mg, 180 mg to 240 mg, 190 mg to 240 mg, or 200 to 240 mg. In some cases, the recombinant apyrase protein may be administered at a dose of 50 mg to 200 mg, 60 mg to 200 mg, 70 mg to 200 mg, 80 mg to 200 mg, 90 mg to 200 mg, 100 mg to 200 mg, 110 mg to 200 mg, 120 mg to 200 mg, 130 mg to 200 mg, 140 mg to 200 mg, 150 mg to 200 mg, 160 mg to 200 mg, 170 mg to 200 mg, or 180 mg to 200 mg. In some cases, the recombinant apyrase protein may be administered at a dose of 50 mg to 180 mg, 60 mg to 180 mg, 70 mg to 180 mg, 80 mg to 180 mg, 90 mg to 180 mg, 100 mg to 180 mg, 110 mg to 180 mg, 120 mg to 180 mg, 130 mg to 180 mg, 140 mg to 180 mg, 150 to 180 mg, or 160 to 180 mg. No significant adverse events observed at these doses, with higher doses expected to see a greater therapeutic (e.g., cardioprotective) effect.

In some cases, the recombinant apyrase protein may be administered to the patient at a dose that is less than 180 mg. As demonstrated herein, while a dose of 180 mg or greater AZD3366 did not elicit any significant adverse events, there was an increase in capillary bleeding time (CBT) measured in heathy volunteers. Although CBT is not a determinative marker for bleeding in a clinical setting, it may be indicative of increased bleeding. For example, the recombinant apyrase protein may be administered to the patient at a dose of 40 mg to 170 mg, 50 mg to 170 mg, 60 mg to 170 mg, 70 mg to 170 mg, 80 mg to 170 mg, 90 mg to 170 mg, 100 mg to 170 mg, 110 mg to 170 mg, 120 mg to 170 mg, 130 mg to 170 mg, 140 mg to 170 mg, 150 mg to 170 mg, or 160 mg to 170 mg. As another example, the recombinant apyrase protein may be administered to the patient at a dose of 40 mg to 160 mg, 50 mg to 160 mg, 60 mg to 160 mg, 70 mg to 160 mg, 80 mg to 160 mg, 90 mg to 160 mg, 100 mg to 160 mg, 110 mg to 160 mg, 120 mg to 160 mg, 130 mg to 160 mg, 140 mg to 160 mg, or 150 mg to 160 mg. As a further example, the recombinant apyrase protein may be administered to the patient at a dose of 40 mg to 150 mg, 50 mg to 150 mg, 60 mg to 150 mg, 70 mg to 150 mg, 80 mg to 150 mg, 90 mg to 150 mg, 100 mg to 150 mg, 110 mg to 150 mg, 120 mg to 150 mg, or 130 mg to 150 mg. As a yet further example, the recombinant apyrase protein may be administered to the patient at a dose of 40 mg to 140 mg, 50 mg to 140 mg, 60 mg to 140 mg, 70 mg to 140 mg, 80 mg to 140 mg, 90 mg to 140 mg, 100 mg to 140 mg, 110 mg to 140 mg, 120 mg to 140 mg, or 130 mg to 140 mg.

In some cases, the recombinant apyrase protein may be administered at a dose that is greater than 90 mg. As demonstrated herein, there was a dose dependent duration relating to platelet aggregation inhibition attributed to AZD3366, with doses greater than 90 mg leading to significantly longer lasting platelet aggregation inhibition effect. For example, the recombinant apyrase protein may be administered to the patient at a dose of 100 mg to 240 mg, 100 mg to 230 mg, 100 mg to 220 mg, 100 mg to 210 mg, 100 mg to 200 mg, 100 mg to 190 mg or 100 mg to 180 mg. It will also be understood that the greater than 90 mg dose can be combined the less than 180 mg dose described above. Hence, in some cases, the recombinant apyrase protein is administered to the patient at a dose of 100 mg to 170 mg, 100 mg to 160 mg, or 100 mg to 150 mg.

Furthermore, without wishing to be bound by theory, by combining the in vivo pharmacokinetic and safety data obtained from the in-human trial with pre-clinical efficacy data using animal models, it is believed that doses of AZD3366 between 100 mg and 140 mg are likely to represent an optimal range between achieving efficacious therapeutic effect (e.g. cardioprotective and/or anti-thrombotic) without compromising patient safety. In particular, this dose range is based on achieving similar plasma exposure in humans as was achieved in the pig animal studies when significant improvements on infarct size were seen.

Hence, in some cases, the recombinant apyrase is administered to the patient at a dose of 100 mg to 140 mg, 100 mg to 135 mg, 100 mg to 130 mg, 105 mg to 140 mg, 105 mg to 135 mg, 105 mg to 130 mg, 110 mg to 140 mg, 110 mg to 135 mg, or 110 mg to 130 mg. In some cases, the recombinant apyrase is administered to the patient at a dose of 105 mg to 125 mg, i.e. at any one of 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg and 125 mg. In some cases, the recombinant apyrase is administered to the patient at a dose of 110 mg to 120 mg, i.e. at any one of 110 mg, 111 mg, 112 mg, 113 mg, 114 mg, 115 mg, 116 mg, 117 mg, 118 mg, 119 mg and 120 mg. In one example, the recombinant apyrase is administered to the patient at dose of 110 mg. In another example, the recombinant apyrase is administered to the patient at dose of 115 mg. In a further example, the recombinant apyrase is administered to the patient at dose of 120 mg.

The precise dosage requirements of recombinant apyrase proteins may vary depending on age, race, weight, height, gender, duration of treatment, methods of administration, biological activity of recombinant apyrase protein, and severity of condition or other clinical variables. While exemplary doses are provided above, other effective dosages within the ranges disclosed herein may be determined by a skilled physician or other skilled medical personnel.

As described above, the recombinant apyrase is typically administered as a flat dose, i.e. a dose that is not based on the patient's individual weight. Alternatively, the recombinant apyrase may be administered at a dose that is calculated based on the patient's weight in kilograms (kg). For example, a flat dose of 120 mg corresponds to 2 mg/kg in a subject with 60 kg body weight.

In some embodiments, the patient being treated has a body weight of between 50 and 100 kg. In some embodiments, the patient being treated has a body mass index (BMI) of between 18 and 30 kg/m².

In some embodiments, the patient being treated is a Chinese or Japanese subject. A Chinese subject is a male or female Chinese person for whom both parents and all grandparents are Chinese and not lived outside of China for more than 10 years. A Japanese subject is a male or female Japanese person for whom both parents and all grandparents are Japanese and not lived outside of China for more than 10 years.

The recombinant apyrase protein may be administered as a pharmaceutical composition comprising the recombinant apyrase protein and a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation. Such diluents and excipients may be comprised of neutral buffered saline solution, antioxidants (for example ascorbic acid), low molecular weight polypeptides (for example polypeptides <10 amino acids) amino acids, carbohydrates (for example, glucose, dextrose, sucrose, or dextrans), chelating agents such as EDTA, stabilizers (such as glutathione). Additionally, co-substrates for the recombinant apyrase proteins, for example, calcium (Ca²⁺) may be administered at time of dosage for maximal activity of the enzyme. Such carriers and diluents are selected to be nontoxic to the patient at recommended dosages and concentrations.

### Combination Treatment

In some of the therapeutic methods described herein, the recombinant apyrase protein is administered in conjunction with the P2Y₁₂ inhibitor and/or aspirin. Use of the term “in conjunction” is this context is intended to mean that following administration (e.g. within 30 minutes, or within an hour, or within 2 hours, or within 3 hours), both the P2Y₁₂ inhibitor and/or aspirin (and/or a metabolite thereof) and recombinant apyrase protein are bioavailable (i.e. have an active effect) in the blood stream of the patient. In animal models, administration of certain doses of the recombinant protein AZD3366 causes it to become active within 5 minutes and does not return to baseline for 3 to 4 weeks, whereas it typically takes longer for P2Y₁₂ inhibitors to become active following administration. For example, maximum activity of the P2Y₁₂ inhibitor ticagrelor is not normally observed until around 2 hours after a dose and this is maintained for more than 8 hours.

The P2Y₁₂ inhibitor disclosed herein may be selected from a list consisting of: ticagrelor, clopidogrel, ticlopidine, prasugrel, and cangrelor. Reference herein to P2Y₁₂ inhibitor includes any of these compounds as well as any metabolites, e.g. active metabolites. In some embodiments, the P2Y₁₂ inhibitor may be selected from a list consisting of: ticagrelor, clopidogrel, ticlopidine and prasugrel, e.g. selected from the list consisting of ticagrelor, clopidogrel and prasugrel. In some embodiments, the P2Y₁₂ inhibitor may be ticagrelor or clopidogrel. In some embodiments, the P2Y₁₂ inhibitor is ticagrelor.

Ticagrelor [(1S,2S,3R,5S)-3-[7-[[(1R,2S)-2-(3,4-difluorophenyl)cyclopropyl]amino]-5-(propylthio)-3H-1,2,3-triazolo[4,5-d]pyrimidin-3-yl]-5-(2-hydroxyethoxy)-1,2-cyclopentanediol] is a reversibly binding oral P2Y(12) receptor antagonist in development for the prevention of thrombotic events in patients with acute coronary syndromes. It has the following chemical structure:

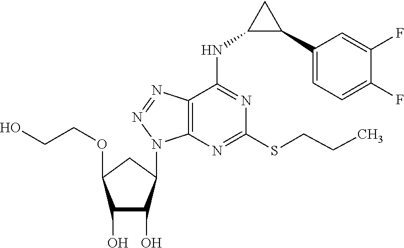

Ticagrelor is the active ingredient in the drug product known as BRILINTA® (or BRILIQUE in Europe) which has been approved for use in multiple jurisdictions including the USA and Europe. Ticagrelor is currently marketed in the form of 60 mg and 90 mg immediate release tablets. WO 2008/024045 discloses certain pharmaceutical formulations containing ticagrelor for oral administration. WO 2017/182589 discloses rapidly disintegrating oral dosage forms of ticagrelor.

Ticagrelor is typically rapidly absorbed after oral administration. Unlike clopidogrel and prasugrel, ticagrelor is not a prodrug and does not require metabolic activation for activity. Still, ticagrelor is extensively metabolised, with ticagrelor and its active and approximately equipotent metabolite (AR-C124910XX), comprising the major circulating components in the plasma. Plasma concentrations of ticagrelor and its active metabolite increase in a dose-dependent manner; peak concentrations achieved within approximately 1.5 and 2.5 hours, respectively. Maximum inhibition of platelet aggregation is observed approximately 2 hours after a dose and this is maintained for more than 8 hours after a dose. The mean elimination half-lives for ticagrelor and its active metabolite are described in the drug label as 7 hours and 9 hours, respectively. Following discontinuance, platelet activity returns to baseline after 5 days.

In some embodiments, ticagrelor is administered orally as a 180 mg loading dose in conjunction with the recombinant apyrase protein. Ticagrelor may be administered in an orodispersible tablet (ODT) form, e.g. as described in WO 2017/182589. One or more subsequent maintenance doses may be administered after the loading dose, e.g. without the recombinant apyrase protein. As described below, following an initial loading dose of 180 mg, the prescribing information for ticagrelor describes administering a maintenance dose of 90 mg twice daily during the first year after an ACS event and after one year administering 60 mg twice daily. The one or more subsequent maintenance doses may comprise twice daily doses of 90 mg of ticagrelor, or twice daily doses of 60 mg of ticagrelor. The prescribing information further describes administering ticagrelor with daily maintenance doses of aspirin of 75-100 mg. Accordingly, the one or more subsequent maintenance doses may further comprise administering daily doses of aspirin of 75-100 mg.

The P2Y₁₂ inhibitor may be clopidogrel. Clopidogrel is typically administered via oral route. In some embodiments, clopidogrel is administered as a 300 mg or 600 mg loading dose in conjunction with the recombinant apyrase protein. One or more subsequent maintenance doses may comprise about 75 mg of clopidogrel and may be administered after the loading dose, e.g. without the recombinant apyrase protein. As with ticagrelor above, the maintenance doses of clopidogrel may be administered daily doses of aspirin of 75-100 mg.

Clopidogrel is a prodrug and requires metabolic activation for its activity. Peak plasma concentrations of the active metabolite occur approximately 30-60 minutes following an oral dose, with dose-dependent platelet aggregation inhibition observed in around 2 hours following administration. Following oral administration of a single dose, dose-dependent platelet aggregation inhibition can be observed in 2 hours. Clopidogrel has an elimination half-life of approximately 6 hours following a single dose of 75 mg, whilst its active metabolite has an elimination half-life of approximately 30 minutes. After discontinuance, platelet aggregation and bleeding times gradually return to baseline in about 5 days.

The P2Y₁₂ inhibitor may be prasugrel. Prasugrel is typically administered via oral route. In some embodiments, prasugrel is administered as a 60 mg loading dose in conjunction with the recombinant apyrase protein. One or more subsequent maintenance doses may comprise about 5 mg or 10 mg of prasugrel and may be administered after the loading dose, e.g. without the recombinant apyrase protein. As with ticagrelor above, the maintenance doses of prasugrel may be administered with daily doses of aspirin of 75-100 mg.

Prasugrel is a prodrug and rapidly metabolised to a pharmacologically active metabolite. Peak plasma concentrations of the active metabolite occur approximately 30 minutes after dosing. It has an elimination half-life of about 7.4 hours.

The P2Y₁₂ inhibitor may be ticlopidine. Ticlopidine is typically administered via oral route. In some embodiments, ticlopidine is typically administered via oral route. In some embodiments, ticlopidine is administered as a 500 mg loading dose in conjunction with the recombinant apyrase protein. One or more subsequent maintenance doses may comprise about 250 mg of ticlopidine and may be administered after the loading dose, e.g. without the recombinant apyrase protein. As with ticagrelor above, the maintenance doses of ticlopidine may be administered daily doses of aspirin of 75-100 mg.

Peak plasma levels of ticlopidine are typically observed around 2 hours after oral administration. Half-life following a single dose ranges from 7 to 13 hours. Half-life following repeated dosing is about 4 to 5 days.

The P2Y₁₂-receptor inhibitor may be cangrelor. Cangrelor may be administered intravenously as a bolus, as a continuous infusion, or as a bolus followed by a continuous infusion. In some embodiments, cangrelor is administered as a 30 μg/kg intravenous bolus followed immediately by 4 μg/kg per minute intravenous infusion.

Cangrelor rapidly reaches steady state plasma levels and platelet aggregation inhibition within 30 min of onset of infusion and the plasma half-life is short, being approximately less than 9 min. Maximal platelet inhibition is achieved within 15 min. The elimination half-life of cangrelor is about 3-6 minutes and platelet responses typically return to baseline within 15 minutes of discontinuation.

The P2Y₁₂ inhibitor may be administered as a pharmaceutical composition comprising the P2Y₁₂ inhibitor and a pharmaceutically acceptable carrier or diluent.

It is not always necessary for both the recombinant apyrase protein and the P2Y₁₂ inhibitor to be physically administered at the same time in order for the recombinant apyrase protein to administered in conjunction with the P2Y₁₂ inhibitor. Rather, the one of the recombinant apyrase protein and P2Y₁₂ inhibitor may be administered first, followed by the other agent being administered later (e.g. an hour or more later) provided that following administration both the recombinant apyrase protein and P2Y₁₂ inhibitor are bioavailable in the blood stream of the patient.

Furthermore, some patients exhibiting an ischemic event may already be regularly administering a P2Y₁₂ inhibitor, e.g. as part of a maintenance doses following a previous ischemic event. Such patients are referred to herein as “currently undergoing treatment with a P2Y₁₂ inhibitor”. For example, 90 mg doses of ticagrelor are typically administered twice daily as part of maintenance doses. In such patients that are administered with the recombinant apyrase protein according to the method herein, it may not always be necessary to administer another dose of the P2Y₁₂ inhibitor because the inhibitor (and/or a metabolite thereof) is still considered bioactive in the blood stream of the patient. Alternatively, a reduced dose than the normal loading dose of the P2Y₁₂ inhibitor may be administered in order to top up the levels of bioactive P2Y₁₂ inhibitor in the blood stream. E.g. in the case of ticagrelor, a dose of 60 mg or 90 mg or 150 mg may be administered if ticagrelor is still bioactive in the patient's blood stream, as opposed to a typical loading dose of 180 mg. Effective dosages may be determined by a skilled physician or other skilled medical personnel. In some embodiments, even if the patient is currently undergoing treatment with a P2Y₁₂ inhibitor, the method still comprises administering the P2Y₁₂ inhibitor (e.g. a loading dose) to the patient.

A P2Y₁₂ inhibitor may still be considered bioactive in the blood stream in the patient if last dose of the inhibitor was administered within a time period corresponding to the mean elimination half-life of the P2Y₁₂ inhibitor, more than twice the mean elimination half-life of the P2Y₁₂ inhibitor, or more than three times the mean elimination half-life of the P2Y₁₂ inhibitor, or more than five times the mean elimination half-life of the P2Y₁₂ inhibitor. For example, in the case of ticagrelor the mean elimination half-life is 7 hours for ticagrelor and 9 hours for its active metabolite. Accordingly, ticagrelor may be considered bioactive in the blood stream in the patient if the last dose was within the last 9 hours, within the last 18 hours, within the last 27 hours, within the last 36 hours, or within the last 45 hours. Alternatively, the P2Y₁₂ inhibitor may be considered bioactive in the blood stream until activity returns to baseline following discontinuance of dosing, which in the case of ticagrelor is after 5 days.

Other patients exhibiting an ischemic event may not have previously been administered a P2Y₁₂ inhibitor, or may have discontinued previous P2Y₁₂ inhibitor administration, e.g. such that the P2Y₁₂ inhibitor is no longer considered bioactive in the patient's blood stream. These patients may be referred to as “naïve” patients. In naïve patients, in order to administer the recombinant apyrase protein in conjunction with the P2Y₁₂ inhibitor, it is necessary for the method to comprise a step of administering the P2Y₁₂ inhibitor to the patient.

Administration of the P2Y₁₂ inhibitor described herein will depend on the particular P2Y₁₂ inhibitor being used. For example, ticagrelor, clopidogrel, ticlopidine and prasugrel are typically administered to patients in a pharmaceutically acceptable oral dosage form, while cangrelor is typically administered to patients via intravenous injection.

The recombinant apyrase protein and P2Y₁₂ inhibitor may be administered as a combined formulation, e.g. via intravenous injection. Alternatively, administration of the recombinant apyrase protein and P2Y₁₂ inhibitor to the patient may be simultaneous or sequential. Simultaneous administration as used herein refers to the administration of both the recombinant apyrase protein and P2Y₁₂ inhibitor to the patient at essentially the same time (e.g. within 10 minutes, within 5 minutes, or within 1 minute of each other), optionally via different administration routes. For example, intravenous injection of the recombinant apyrase protein within 1 minute of the P2Y₁₂ inhibitor being provided by oral administration would be considered simultaneous administration.

Where sequential administration is used, the recombinant apyrase protein and P2Y₁₂ inhibitor may be administered within 18 hours, 12 hours, 6 hours, or 2 hours of each other. In some embodiments the recombinant apyrase protein is administered first, followed by the sequential administration of the P2Y₁₂ inhibitor. As exemplified herein, the recombinant apyrase protein may be administered to the patient and the P2Y₁₂ inhibitor (e.g. ticagrelor) administered within 2 hours (e.g. 1 hour 40 minutes) later. In other embodiments the P2Y₁₂ inhibitor is administered first, followed by the sequential administration of the recombinant apyrase protein.

In some embodiments, the recombinant apyrase protein and the P2Y₁₂ inhibitor are both administered to the patient before carrying out surgical reperfusion therapy (e.g. PCI). In other embodiments, the recombinant apyrase protein is administered to the patient before carrying out surficial reperfusion therapy (e.g. PCI) and the P2Y₁₂ inhibitor is administered shortly after, e.g. within 6 hours, within 4 hours, within 2 hours, or within 1 hour of the surgical reperfusion therapy being performed. In some embodiments, the recombinant apyrase protein and P2Y₁₂ inhibitor remain bioavailable in the patient's blood stream during surgical reperfusion therapy (e.g. PCI).

The disclosure includes embodiments where any of the timings described above regarding i) simultaneous or sequential administration of the recombinant apyrase polypeptide and the P2Y₁₂ inhibitor; ii) timing of administration in relation to the onset of the ischemic event; and iii) timing of administration in relation to the surgical reperfusion therapy (e.g. PCI) are combined. For example, the method may comprise administering the recombinant apyrase protein within 6 hours of the onset of the ischemic event, wherein the method further comprises carrying out surgical reperfusion therapy (e.g. PCI) on the patient in less than 12 hours, or less than 6 hours, and wherein the P2Y₁₂ inhibitor is administered to the patient within 6 hours of administering the recombinant apyrase protein, optionally wherein both the recombinant apyrase protein and the P2Y₁₂ inhibitor are administered to the patient before carrying out surgical reperfusion therapy (e.g. PCI).

Any of the methods described herein may further comprise administering aspirin to the patient. Aspirin is typically administered as a separate formulation to the P2Y₁₂ inhibitor and recombinant apyrase protein and is administered simultaneously or sequentially to either one or both of the P2Y₁₂ inhibitor and recombinant apyrase protein. In some embodiments, aspirin is administered within 24 hours, 18 hours, 12 hours, 6 hours, 2 hours, 1 hour or within 30 minutes of administering the P2Y₁₂ inhibitor. In some embodiments, aspirin is administered within 24 hours, 18 hours, 12 hours, 6 hours, 2 hours, 1 hour or within 30 minutes of administering the recombinant apyrase protein. As exemplified herein, the aspirin may be administered to the patient, followed by the recombinant apyrase protein administered 2 hours later and then the P2Y₁₂ inhibitor (e.g. ticagrelor) administered within 2 hours (e.g. 1 hour 40 minutes) of the recombinant apyrase protein administration.

In some embodiments, aspirin is administered simultaneously with the P2Y₁₂ inhibitor or recombinant apyrase protein. In some embodiments, the aspirin is administered to the patient in conjunction with the P2Y₁₂ inhibitor or 18 hours or less, or 12 hours or less, or 6 hours or less, or 4 hours or less, or 2 hours or less, or 1 hour or less, or even 30 minutes or less after onset of the ischemic event (e.g. acute coronary syndrome such as STEMI).

Aspirin may be administered to the patient at a dose between 50 mg to 325 mg, e.g. 50 mg, 75 mg, 100 mg, 125 mg, 150 mg, 162 mg, 175 mg, 200 mg, 225 mg, 250 mg, 275 mg, 300 mg, 324 mg, 325 mg or 350 mg. In some embodiments, aspirin is administered to the patient at a dose between 50 mg and 200 mg, or between 100 mg and 200 mg, e.g. 162 mg. In some embodiments, aspirin is administered to the patient at a loading dose between 200 mg and 350 mg, or between 250 and 325 mg, e.g. 300 mg, 324 mg or 325 mg. In some embodiments, aspirin is administered to the patient at a loading dose of 324 mg. In some embodiments, aspirin is administered to the patient at a maintenance dose between 75 mg and 150 mg, or between 75 mg and 100 mg, e.g. 81 mg.

The method may comprise administering the recombinant apyrase proteins as a single effective dose in conjunction with a suitable dose (e.g. loading dose) of the P2Y₁₂ inhibitor, and optionally aspirin if present. Whilst only a single effective dose of the recombinant apyrase protein is typically used, the method may further comprise administering one or more oral doses of the P2Y₁₂ inhibitor periodically and subsequent to the loading dose, as part of a chronic or maintenance treatment. For example, the P2Y₁₂ inhibitor may be administered once or twice a day, for weeks, months or even years following the initial loading dose, e.g. at the maintenance doses described above. The maintenance doses of P2Y₁₂ inhibitor may be administered with aspirin, as is known in the art (referred to as dual anti-platelet therapy, or DAPT). Chronic or maintenance treatment of P2Y₁₂ inhibitors following an ischemic event is known in the art and appropriate doses and timing can be determined by a skilled physician or other skilled medical personnel.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof. While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—A Phase 1, Randomized, Single-blind, Placebo-controlled Study to Assess the Safety, Tolerability, Pharmacokinetics and Pharmacodynamics of AZD3366 in Healthy Men and Women of Non-Childbearing Potential

### Study Overview

This first-in-human (FiH) study was conducted to provide data on safety, tolerability, pharmacokinetics (PK), and pharmacodynamics (PD) of AZD3366 in healthy male and female subjects. This study has the ClinicalTrial.gov identifier: NCT04588727

This study was conducted in healthy male and female (of non-childbearing potential) subjects age 18 to 55 years with a body weight of at least 50 kg and no more than 100 kg inclusive and a body mass index (BMI) range of 18 to 30 kg/m², inclusive.

Three populations (healthy subjects, healthy Japanese subjects and healthy Chinese subjects) were enrolled into this study. The healthy subject population contained healthy male and female (of non-childbearing potential) subjects that did not have origins in any of the original peoples of the Far East, Southeast Asia, or the Indian subcontinent. The healthy Japanese population contained healthy male and female (of non-childbearing potential) Japanese subjects for whom both parents and all grandparents were Japanese and not lived outside of Japan for more than 10 years. The healthy Chinese population contained healthy male and female (of non-childbearing potential) Chinese subjects for whom both parents and all grandparents were Chinese and not lived outside of China for more than 10 years.

Part A of the study was a randomized, single-blind, placebo-controlled design to assess the safety, tolerability, PK, and PD (capillary bleeding time [CBT] and inhibition of platelet aggregation) of an intravenous (IV) administration of single ascending doses (SAD) of AZD3366 in healthy subjects, healthy Japanese subjects and healthy Chinese subjects.

Part B of the study was a randomized, single-blind, parallel-group placebo-controlled design to investigate the safety, tolerability, and PD (CBT and inhibition of platelet aggregation) of a single IV dose of AZD3366 with concomitant administration of ticagrelor and acetylsalicylic acid (ASA) in healthy subjects.

Co-medication with ASA and ticagrelor was chosen based on the Standard of Care anti-platelet treatment regimen in patients with myocardial infarction.

### Primary Objectives

The primary objectives of this study were:

### Part A:

To investigate the safety and tolerability of intravenous (IV) administration of SAD of AZD3366 in healthy subjects, healthy Japanese subjects and healthy Chinese subjects

### Part B:

To investigate the safety and tolerability of a single IV administration of one dose level (160 mg) of AZD3366 in healthy subjects with concomitant loading dose and repeated dosing of ticagrelor and ASA.

### Secondary Objectives

The secondary objectives of this study were:

### Part A:

To characterize the PK of AZD3366 following IV administration of single doses of AZD3366 in healthy subjects, healthy Japanese subjects and healthy Chinese subjects.
To characterize the PD of AZD3366 following IV administration of single doses of AZD3366 with respect to inhibition of platelet aggregation Light Transmission Aggregometry [LTA] and CBT in healthy subjects, healthy Japanese subjects and healthy Chinese subjects.
To explore immunogenicity following IV administration of AZD3366.

### Part B:

To study the plasma exposure and characterize the PD of AZD3366 with respect to inhibition of platelet aggregation LTA and CBT, following IV administration of AZD3366 at one dose level in healthy subjects with concomitant loading dose and repeated dosing of ticagrelor and ASA.
To study the effect of AZD3366 on the PK of ticagrelor.
To explore immunogenicity following IV administration of AZD3366.

### Dose and Treatment Regimens

### Part A:

7 cohorts of 8 subjects received a single short IV infusion of AZD3366 (2-640 mg [n=6]) or placebo (n=2). A further 3 cohorts of 5 healthy Japanese/1 cohort of 8 healthy Chinese subjects were included within this dose range. Overall, 103 subjects were randomized; mean age 37.2 (+9) years, 100 were male.

The first cohort received 2 mg AZD3366 or placebo. Cohorts 2-7 received 10 mg, 30 mg, 90 mg, 180 mg, 360 mg and 640 mg, respectively.

### Part B:

In Part B, a separate group of 24 subjects received a single IV dose of 160 mg AZD3366 or placebo in combination with aspirin and ticagrelor (n=12) or aspirin and ticagrelor alone (n=12).

Healthy volunteers were administered a loading dose of 324 mg ASA and 2 hours later administered a single IV dose of AZD3366 or placebo (t=0). 1 hour 40 minutes after administering AZD3366 or placebo the healthy volunteers were administered a loading dose of 180 mg ticagrelor. 90 mg ticagrelor was administered every 12 hours following the loading dose (t=25 h 40 m, t=37 h 40 m, t=49 h 40 m). ASA was administered daily at t=25 h 40 m and t=49 h 40 m). Capillary bleeding time was monitored at t=0 h 10 m, t=3 h 40 m and t=51 h 40 m.

### Measurements and Methods of Assessment

Light Transmission Aggregometry (LTA) to Measure ADP-Induced Platelet Response after Administration of AZD3366 to Healthy Volunteers

ADP-induced platelet aggregation was quantified in Platelet Rich Plasma (PRP) using an LTA assay on a CHRONO-LOG® 490 4+ aggregation system with AGGGRO/LINK® interface. PRP was prepared by centifugation of lithium heparin-anticoagulated blood sample at 100-170 g for 15 minutes followed by removal of the supernatant to a fresh polyproylene tube. For setting 100% baseline in the LTA assay, Platelet Poor Plasma (PPP) was prepared by centrifugation of blood sample at 1500-2400 g for 20 minutes followed by transfer of the supernatant to a fresh polyproylene tube. To stimulate platelet aggregation, ADP at 5 or 20 μmol/L was added to PRP and PPP preparations in separate cuvettes containing a magnetic stirrer, and aggregation was followed for a minimum of 5 minutes at 37° C. with constant stirring. Data was recorded as % Amplitude (aggregation response in PRP sample expressed as a % of the PPP response) and Area Under the Curve (AUC). For each AZD3366 dose and timepoint, the inhibition of platelet aggregation was calculated relative to the pre-dose level of platelet aggregation.

Capillary Bleeding Time (CBT) Assessment after Administration of AZD3366 to Healthy Volunteers

CBT was evaluated by making a horizontal incision in the forearm and monitoring the time for bleeding to stop. A syphgmomanometer cuff was placed on the upper arm and inflated to 40 mmHg. A standardised incision was then made with a Surgicutt® device and bleeding was monitored at 30 second intervals by touching a filter paper (Surgicutt® Bleeding Time Blotting paper) over the bleed, but no closer than 0.5 mm from the incision to avoid disturbing the formation of the platelet plug. Bleeding was followed for up to 90 or 180 minutes and adjudicated to have stopped when blood no longer stained the filter paper. If bleeding had not stopped by the 90 or 180 minute cut-off, bleeding was adjudicated to be still ongoing at this timepoint. Following completion of the CBT analysis, the cuff was removed and the incision site was cleansed with an antiseptic swab.

### Part A Results

AZD3366 treatment alone did not result in any clinically relevant safety or tolerability findings. Comparable rates of adverse events (AEs) in the AZD3366 and placebo groups were observed, with no significant increase in bleeding events. A summary of AE results for the healthy volunteers treated with AZD3366 (across all doses) or placebo is provided in the following table:

Number (%) of subjects

| Adverse events by System | Total AZD3366 | Pooled Placebo |
|---|---|---|
| Organ Class | (N = 42) | (N = 14) |
| Subjects with any AE | 16 (38.1) | 5 (35.7) |
| General disorders and site | 7 (16.7) | 2 (14.3) |
| conditions | | |
| Musculoskeletal and connective | 4 (9.5) | 2 (14.3) |
| tissue disorders | | |
| Gastrointestinal disorders | 3 (7.1) | 1 (7.1) |
| Nervous systems disorders | 3 (7.1) | 1 (7.1) |
| Cardiac disorders | 2 (4.8) | 0 |
| Skin and subcutaneous tissue | 2 (4.8) | 2 (14.3) |
| disorders | | |
| Vascular disorders | 2 (4.8) | 0 |

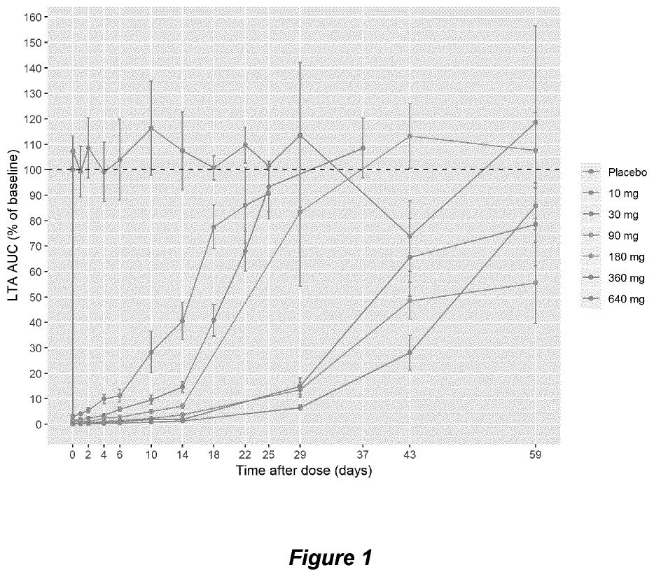

Peak AZD3366 plasma concentrations were reached within 0.5 h post-dosing, followed by monophasic or biphasic decline with a terminal PK half-life of approximately 140 h. Complete ADP-stimulated platelet aggregation inhibition was reached within 10 mins post-dosing; duration ranged from 4 h at 2 mg to ˜35 days at 640 mg (FIG. 1).

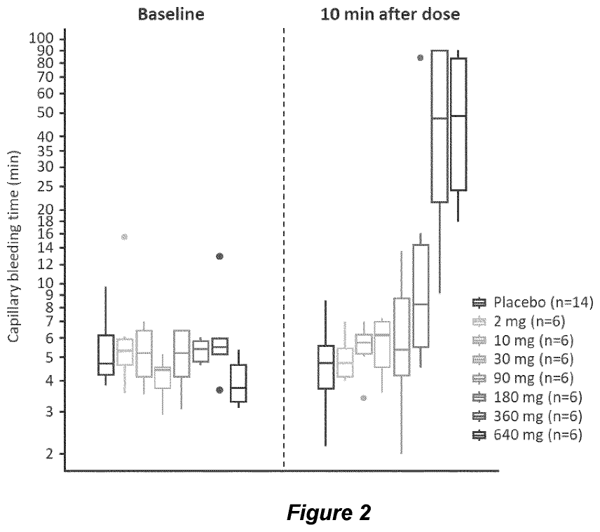

In the single treatment of AZD3366, no increase in capillary bleeding time (CBT) was observed up to the 90 mg dose level while a significant increase was observed at the 180 mg dose and above (FIG. 2).

### Part B Results

AZD3366 treatment in combination with aspirin and ticagrelor (AZD3366+DAPT) was safe and well tolerated. As with part A, there were comparable rates of adverse events in the AZD3366 and placebo groups, with no significant increase in bleeding events. A summary of AE results for the two groups is provided in the following table:

Number (%) of subjects

| Adverse events by System | AZD3366 + DAPT | Placebo + DAPT |
|---|---|---|
| Organ Class | (N = 12) | (N = 12) |
| Subjects with any AE | 2 (16.7) | 7 (58.3) |
| Injury, poisoning and procedural | 1 (8.3) | 1 (8.3) |
| complications | | |
| Skin and subcutaneous tissue | 1 (8.3) | 1 (8.3) |
| disorders | | |
| Gastrointestinal disorders | 0 | 1 (8.3) |
| General disorders and | 0 | 5 (41.7) |
| administration site conditions | | |
| Nervous system disorders | 0 | 1 (8.3) |
| Psychiatric disorders | 0 | 2 (16.7) |

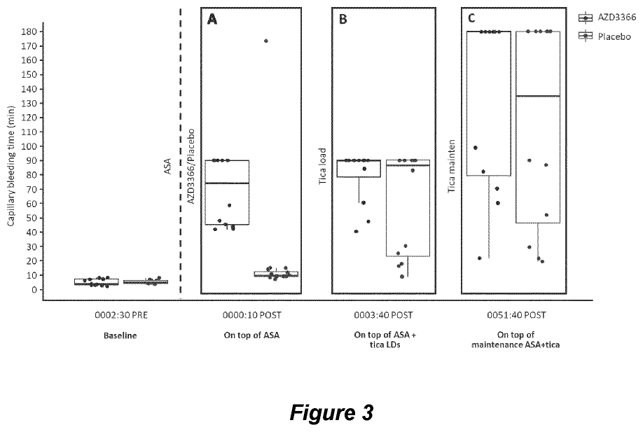

There was an increase in CBT in the AZD3366 group compared to placebo when loading doses of ASA and ticagrelor were given but that increase levelled off when healthy volunteers continued with maintenance doses of DAPT (FIG. 3).

### CONCLUSIONS

AZD3366 alone or in combination with aspirin and ticagrelor was generally safe, well tolerated, and achieved complete platelet inhibition with a dose-dependent duration.

### REFERENCES

A number of publications are cited above in order to more fully describe and disclose the invention and the state of the art to which the invention pertains. Full citations for these references are provided below. The entirety of each of these references is incorporated herein.
Asaria, P., Elliott, P., Douglass, M., Obermeyer, Z., Soljak, M., Majeed, A., & Ezzati, M. (2017). Acute myocardial infarction hospital admissions and deaths in England: a national follow-back and follow-forward record-linkage study. The Lancet. Public health, 2(4), e191-e201. https://doi.org/10.1016/S2468-2667(17)30032-4
Ibanez, B., James, S., Agewall, S., Antunes, M. J., Bucciarelli-Ducci, C., Bueno, H., Caforio, A., Crea, F., Goudevenos, J. A., Halvorsen, S., Hindricks, G., Kastrati, A., Lenzen, M. J., Prescott, E., Roffi, M., Valgimigli, M., Varenhorst, C., Vranckx, P., Widimský, P., & ESC Scientific Document Group (2018). 2017 ESC Guidelines for the management of acute myocardial infarction in patients presenting with ST-segment elevation: The Task Force for the management of acute myocardial infarction in patients presenting with ST-segment elevation of the European Society of Cardiology (ESC). European heart journal, 39(2), 119-177. https://doi.org/10.1093/eurheartj/ehx393
Jernberg, T., Hasvold, P., Henriksson, M., Hjelm, H., Thuresson, M., & Janzon, M. (2015). Cardiovascular risk in post-myocardial infarction patients: nationwide real world data demonstrate the importance of a long-term perspective. European heart journal, 36(19), 1163-1170. https://doi.org/10.1093/eurheartj/ehu505
Moeckel D, Jeong S S, Sun X, Broekman M J, Nguyen A, Drosopoulos J H, Marcus A J, Robson S C, Chen R, Abendschein D. (2014) Optimizing human apyrase to treat arterial thrombosis and limit reperfusion injury without increasing bleeding risk. Sci Transl Med., 6(248):248ra105. doi: 10.1126/scitranslmed.3009246.
Robson, S. C., Wu, Y., Sun, X., Knosalla, C., Dwyer, K., & Enjyoji, K. (2005). Ectonucleotidases of CD39 family modulate vascular inflammation and thrombosis in transplantation. Seminars in thrombosis and hemostasis, 31(2), 217-233. https://doi.org/10.1055/s-2005-869527
Sun, Guanghua & Zhao, Xiurong & Grotta, James & Savitz, Sean & Chen, Ridong & Aronowski, Jaroslaw. (2011). Apyrase, APT102, Improves the Beneficial Effect of rt-PA In Experimental Thromboembolic Stroke. E302-E302.
Tan, Z., Li, X., Turner, R. C., Logsdon, A. F., Lucke-Wold, B., DiPasquale, K., Jeong, S. S., Chen, R., Huber, J. D., & Rosen, C. L. (2014). Combination treatment of r-tPA and an optimized human apyrase reduces mortality rate and hemorrhagic transformation 6h after ischemic stroke in aged female rats. European journal of pharmacology, 738, 368-373. https://doi.org/10.1016/j.ejphar.2014.05.052
Thygesen, K., Alpert, J. S., Jaffe, A. S., Chaitman, B. R., Bax, J. J., Morrow, D. A., White, H. D., & Executive Group on behalf of the Joint European Society of Cardiology (ESC)/American College of Cardiology (ACC)/American Heart Association (AHA)/World Heart Federation (WHF) Task Force for the Universal Definition of Myocardial Infarction (2018). Fourth Universal Definition of Myocardial Infarction (2018). Circulation, 138 (20), e618-e651. https://doi.org/10.1161/CIR.0000000000000617
Wallentin, L., Becker, R. C., Budaj, A., Cannon, C. P., Emanuelsson, H., Held, C., Horrow, J., Husted, S., James, S., Katus, H., Mahaffey, K. W., Scirica, B. M., Skene, A., Steg, P. G., Storey, R. F., Harrington, R. A., PLATO Investigators, Freij, A., & Thorsen, M. (2009). Ticagrelor versus clopidogrel in patients with acute coronary syndromes. The New England journal of medicine, 361(11), 1045-1057. https://doi.org/10.1056/NEJMoa0904327
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

Sequences

| Identi- | | |
|---|---|---|
| fier | Description | Sequence |
| SEQ ID | Human CD39L3 | MVTVLTRQPCEQAGLKALYRTPTII |
| NO: 1 | amino acid | ALVVLLVSIVVLVSITVIQIHKQEV |
| | sequence | LPPGLKYGIVLDAGSSRTTVYVYQW |
| | (amino acid | PAEKENNTGVVSQTFKCSVKGSGIS |
| | residues | SYGNNPQDVPRAFEECMQKVKGQVP |
| | 49-485 of | SHLHGSTPIHLGATAGMRLLRLQNE |
| | SEQ ID | TAANEVLESIQSYFKSQPFDFRGAQ |
| | NO: 1 | IISGQEEGVYGWITANYLMGNFLEK |
| | underlined) | NLWHMWVHPHGVETTGALDLGGAST |
| | | QISFVAGEKMDLNTSDIMQVSLYGY |
| | | VYTLYTHSFQCYGRNEAEKKFLAM |
| | | LLQNSPTKNHLTNPCYPRDYSISFT |
| | | MGHVEDSLCTVDQRPESYNPNDVIT |
| | | FEGTGDPSLCKEKVASIFDFKACH |
| | | DQETCSFDGVYQPKIKGPFVAFAG |
| | | FYYTASALNLSGSFSLDTENSSTWN |
| | | FCSQNWSQLPLLLPKFDEVYARSYC |
| | | FSANYIYHLFVNGYKFTEETWPQIH |
| | | FEKEVGNSSIAWSLGYMLSLTNQIP |
| | | AESPLIRLPIEPPVFVGTLAFFTAA |
| | | ALLCLAFLAYLCSATRRKRHSEHAF |
| | | DHAVDSD |
| SEQ ID | AZD3366 | EVLPPGLKYGIVLDAGSSGTRVYV |
| NO: 2 | amino acid | YQWPAEKENNTGVVSQTFKCSVKGS |
| | sequence | GISSYGNNPQDVPRAFEECMQKVKG |
| | (N-terminal | QVPSHLHGSTPIHLGATAGMRLLRL |
| | EVLP | QNETAANEVLESIQSYFKSQPFDFR |
| | underlined, | GAQIISGQEEGVYGWITANYLMGNF |
| | R67G and | LEKNLWHMWVHPHGVETTGALDLGG |
| | T69R amino | ASTQISFVAGEKMDLNTSDIMQVSL |
| | acid | YGYVYTLYTHSFQCYGRNEAEKKFL |
| | substitutions | AMLLQNSPTKNHLTNPCYPRDYSIS |
| | bold | FTMGHVEDSLCTVDQRPESYNPND |
| | underline) | VITFEGTGDPSLCKEKVASIFDFKA |
| | | CHDQETCSFDGVYQPKIKGPFVAFA |
| | | GFYYTASALNLSGSFSLDTENSSTW |
| | | NFCSQNWSQLPLLLPKFDEVYARSY |
| | | CESANYIYHLFVNGYKFTEETWPQI |
| | | HFEKEVGNSSIAWSLGYMLSLTNQI |
| | | PAESPLIRLPIEPPV |

1. A method of treating an ischemic event in a patient, the method comprising administering to the patient a therapeutically effective amount of a recombinant apyrase protein,
wherein the recombinant apyrase protein comprises the amino acid sequence set forth as SEQ ID NO: 2, and
wherein the method comprises administering the recombinant apyrase protein to the patient at a dose of 40 mg to 240 mg.

2. A recombinant apyrase protein for use in a method of treating an ischemic event in a patient,
wherein the recombinant apyrase protein comprises the amino acid sequence set forth as SEQ ID NO: 2, and
wherein the method comprises administering the recombinant apyrase protein to the patient at a dose of 40 mg to 240 mg.

3. The method according to claim 1, or recombinant apyrase protein for use according to claim 2, wherein the method comprises administering the recombinant apyrase protein to the patient at a dose of 40 mg to 170 mg.

4. The method according to claim 1, or recombinant apyrase protein for use according to claim 2, wherein the method comprises administering the recombinant apyrase protein to the patient at a dose of 100 mg to 240 mg.

5. The method, or recombinant apyrase protein for use, according to any one of claims 1 to 4, wherein the method comprises administering the recombinant apyrase protein to the patient at a dose of 100 mg to 170 mg.

6. The method, or recombinant apyrase protein for use, according to any one of claims 1 to 5, wherein the method comprises administering the recombinant apyrase protein to the patient at a dose of 100 mg to 140 mg, optionally wherein the recombinant apyrase is administered to the patient at dose of 110 mg, 115 mg or 120 mg.

7. The method, or recombinant apyrase protein for use, according to any one of claims 1 to 6, wherein the ischemic event is an acute coronary syndrome.

8. The method, or recombinant apyrase protein for use, according to claim 7, wherein the ischemic event is ST-segment elevation myocardial infarction (STEMI).

9. The method, or recombinant apyrase protein for use, according to any one of claims 1 to 6, wherein the ischemic event is an acute ischemic stroke.

10. The method, or recombinant apyrase protein for use, according to any one of claims 1 to 9, wherein the recombinant apyrase protein in conjunction with a P2Y₁₂ inhibitor.

11. The method, or recombinant apyrase protein for use, according to claim 10, wherein the P2Y₁₂ inhibitor is selected from a list consisting of: ticagrelor, clopidogrel, ticlopidine, prasugrel, and cangrelor.

12. The method, or recombinant apyrase protein for use, according to claim 11, wherein the P2Y₁₂ inhibitor is ticagrelor, optionally wherein the ticagrelor and is administered at a loading dose of between 60 to 200 mg.

13. The method, or recombinant apyrase protein for use, according to any one of claims 10 to 12, wherein the P2Y₁₂ inhibitor is administered within 2 hours of administering the recombinant apyrase protein to the patient.

14. The method, or recombinant apyrase protein for use, according to any one of claims 10 to 13, wherein the method further comprises administering aspirin to the patient, optionally wherein the aspirin is administered at a loading dose between 250 and 325 mg.

15. The method, or recombinant apyrase protein for use, according to claim 14, wherein the aspirin is administered within 2 hours of administering the recombinant apyrase protein to the patient.

16. The method, or recombinant apyrase protein for use, according to any one of claims 10 to 15, wherein the method further comprises administering maintenance doses of P2Y₁₂ inhibitor and aspirin for at least 1 week following administering of the recombinant apyrase protein to the patient.


SEQUENCE LISTING (2 sequences)

SEQ ID NO: 1 - protein, 529 aa, synthetic construct
MVTVLTRQPCEQAGLKALYRTPTIIALVVLLVSIVVLVSITVIQIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPVFVGTLAFFTAAALLCLAFLAYLCSATRRKRHSEHAFDHAVDSD

SEQ ID NO: 2 - protein, 438 aa, synthetic construct
EVLPPGLKYGIVLDAGSSGTRVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPV